(19) United States
(12) Patent Application Publication
(10) Pub. No.: US 2020/0300235 A1
(43) Pub. Date: Sep. 24, 2020

(54) PERISTALTIC PUMP WITH TWO-PART FLUID CHAMBER AND ASSOCIATED DEVICES, SYSTEMS, AND METHODS

(71) Applicant: Verily Life Sciences LLC, South San Francisco, CA (US)

(72) Inventor: Amir Samakar, Fremont, CA (US)

(21) Appl. No.: 16/820,300

(22) Filed: Mar. 16, 2020

Related U.S. Application Data
(60) Provisional application No. 62/819,905, filed on Mar. 18, 2019.

(51) Int. Cl. F04B 43/00; F04B 43/12; A61F 9/007

(52) U.S. Cl. CPC F04B 43/0045; F04B 43/0072; A61F 9/00709; F04B 43/1238

(57) ABSTRACT

Peristaltic pump assemblies that include two-part fluid chambers are provided. In some embodiments, a pump assembly can include a roller assembly and a two-part fluid chamber comprising a hard outer portion and a flexible membrane coupled to the hard outer portion. The hard outer portion includes a concave or bell-shaped curved surface and a flexible membrane attached to the hard outer portion and extending over the curved surface of the hard outer portion. The bell-shaped groove and the flexible membrane define the fluid channel, and a roller coupled to the fluid chamber and configured to deform the flexible membrane against the bell-shaped groove on the inner surface of the hard outer portion to collapse the fluid channel. The two-part construction of the fluid chamber can decrease the amount of stress experienced by the flexible membrane, thereby increasing the longevity of the fluid chamber.

### CROSS REFERENCE TO RELATED APPLICATIONS

The present application claims priority to and the benefit of U.S. Provisional Patent Application No. 62/819,905, filed Mar. 18, 2019, the entirety of which is incorporated by reference in its entirety.

### TECHNICAL FIELD

The present disclosure relates generally to devices and methods for pumping fluids from a patient and/or delivering pharmaceutical agents to a patient, including peristaltic pump assemblies implantable in a patient for relieving intraocular pressure.

### BACKGROUND

Intraocular pressure (IOP) quantifies the pressure of the aqueous humor inside the eye. Many individuals suffer from disorders, such as glaucoma, that cause chronic heightened IOP. Over time, heightened IOP can cause damage to the optical nerve of the eye, leading to loss of vision. Presently, treatment of glaucoma mainly involves periodically administering pharmaceutical agents to the eye to decrease IOP. These drugs can be delivered by, for example, injection or eye drops. However, the effectiveness of pharmaceuticals can vary greatly from patient-to-patient. Furthermore, effective treatment of glaucoma requires adherence to rigid dosage schedules that can be difficult to follow for some patients.

Another way IOP can be reduced is by removing some of the fluid from inside the patient's eye. However, current devices are not suitable or practical for therapeutic use. For example, current devices do not simultaneously satisfy the desire for small size, low power, and a lifetime of many years before failure. Thus, there remains a need for wearable fluid displacement devices that meet requirements for safety and reliability while being as cost-effective as possible.

### SUMMARY

The present disclosure advantageously describes peristaltic pump assemblies configured to pump fluid from a patient and/or deliver pharmaceutical agents to the patient. In some embodiments, a pump assembly can include a compressing member and a round fluid chamber comprising a hard outer portion and a flexible membrane coupled to the hard outer portion. The compressing member is controlled by a motor to rotate along a circumference to compress the fluid chamber in a circular motion, thereby pumping a fluid through the fluid chamber. The two-part construction of the fluid chamber can decrease the amount of stress experienced by the flexible membrane, thereby increasing the longevity of the fluid chamber.

In one embodiment, a peristaltic pump assembly includes a fluid chamber comprising a fluid channel configured to allow a fluid to pass therethrough, the fluid chamber including a hard outer portion comprising a bell-shaped groove on an inner surface of the hard outer portion and a flexible membrane attached to the hard outer portion and extending over the inner surface of the hard outer portion, wherein the bell-shaped groove and the flexible membrane define the fluid channel, and a roller coupled to the fluid chamber and configured to deform the flexible membrane against the bell-shaped groove on the inner surface of the hard outer portion to collapse the fluid channel.

In some embodiments, the flexible membrane and the bell-shaped groove of the hard outer portion are configured such that a maximum stress experienced by the flexible membrane while being deformed against the bell-shaped groove is below a fatigue limit of the flexible membrane. In some embodiments, the flexible membrane comprises a thickness between 25 um and 150 um. In some embodiments, the thickness of the flexible membrane is 50 um. According to some aspects, the roller comprises a fillet radius that is less than a radius of the bell-shaped groove. In some embodiments, a thickness of the flexible membrane is less than the fillet radius of the roller. In some embodiments, the flexible membrane is attached to the hard outer portion by an adhesive. In one aspect, the flexible membrane is attached to the hard outer portion by a laser weld. In another aspect, the flexible membrane is formed to include a camber. In still another aspect, the flexible membrane comprises silicone rubber. In some embodiments, the hard outer portion comprises an annular shape.

In some embodiments, the bell-shaped curve comprises at least one of a Gaussian curve, a symmetric spline, a sinusoidal curve, or a mirrored biarc. In some embodiments, the bell-shaped curve comprises an inflection point between a concave portion of the bell-shaped curve and a convex portion of the bell-shaped curve. In some embodiments, the flexible membrane further includes a coating positioned over an outer face of the flexible membrane, wherein a coefficient of friction of the coating is less than a coefficient of friction of the outer surface of the flexible membrane.

According to another embodiment of the present disclosure, a method of assembling a peristaltic pump assembly includes assembling a fluid chamber, wherein assembling the fluid chamber comprises: providing a hard outer portion comprising a bell-shaped groove on an inner surface of the hard outer portion; and attaching a flexible membrane to the hard outer portion such that the flexible membrane extends over the inner surface of the hard outer portion, and such that the flexible membrane and the bell-shaped groove of the hard outer portion define a fluid channel; and coupling a roller assembly comprising a roller to the fluid chamber such that the roller is configured to pass over the flexible membrane to deform the flexible membrane against the bell-shaped groove of the hard outer portion.

In some aspects, the flexible membrane and the bell-shaped groove of the hard outer portion are configured such that a maximum stress experienced by the flexible membrane while being deformed against the bell-shaped groove is below a fatigue limit of the flexible membrane. In some embodiments, the method further includes forming a roller fillet comprising a fillet radius that is less than a radius of the bell-shaped groove. In some embodiments, attaching the flexible membrane to the hard outer portion comprises attaching the flexible membrane to the hard outer portion using an adhesive. In some embodiments, attaching the flexible membrane to the hard outer portion comprises attaching the flexible membrane to the hard outer portion using a laser weld. In some embodiments, the method further includes forming the flexible membrane to include a camber.

In some embodiments, the bell-shaped curve comprises at least one of a Gaussian curve, a symmetric spline, a sinusoidal curve, or a mirrored biarc. In some embodiments, the bell-shaped curve comprises an inflection point between a concave portion of the bell-shaped curve and a convex portion of the bell-shaped curve. In some embodiments, the flexible membrane further includes a coating positioned over an outer face of the flexible membrane, wherein a coefficient of friction of the coating is less than a coefficient of friction of the outer surface of the flexible membrane.

According to another embodiment of the present disclosure, a peristaltic pump assembly comprises an annular fluid chamber comprising a hard ring comprising a concave groove on an inner surface of the hard ring and a membrane attached to the hard ring and extending over the inner surface of the hard ring to form a fluid channel comprising a curved cross-section, and a roller assembly coupled to the fluid chamber comprising a roller configured to deform the membrane against the concave groove on the inner surface of the hard ring to collapse the fluid channel.

In some embodiments, the membrane and the concave groove of the hard ring are configured such that a maximum stress experienced by the membrane while being deformed against the concave groove is below a fatigue limit of the membrane. In some embodiments, at least a portion of the concave groove comprises a circular arc.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic view of a micropump system, according to an embodiment of the present disclosure.

FIG. 2 is a diagrammatic schematic view of a micropump assembly, according to an embodiment of the present disclosure.

FIG. 3 is a perspective view of a driver assembly and fluid chamber of a micropump assembly, according to an embodiment of the present disclosure.

FIG. 4 is a perspective view of a micropump assembly, according to an embodiment of the present disclosure.

FIG. 5 is a cross-sectional perspective view of a fluid chamber of a micropump assembly, according to an embodiment of the present disclosure.

FIG. 6 is a perspective view of a driver assembly of a micropump assembly, according to an embodiment of the present disclosure.

FIG. 7 is a cross-sectional perspective view of the driver assembly of FIG. 6, according to an embodiment of the present disclosure.

FIG. 8 is a diagrammatic schematic view of a driver circuit and fluid chamber of a micropump assembly, according to an embodiment of the present disclosure.

FIG. 9 is a diagrammatic schematic view of a micropump assembly, according to an embodiment of the present disclosure.

FIG. 10 is a diagrammatic schematic view of a micropump assembly, according to an embodiment of the present disclosure.

FIG. 11 is a cross-sectional diagrammatic view of a fluid chamber assembly in an uncompressed position, according to an embodiment of the present disclosure.

FIG. 12 is a cross-sectional diagrammatic view of a fluid chamber assembly being compressed by a roller, according to an embodiment of the present disclosure.

FIG. 13 is a graphical view of a fluid chamber assembly being compressed by a roller, according to aspects of the present disclosure.

FIG. 14 is a graphical view of a fluid chamber assembly being compressed by a roller, according to aspects of the present disclosure.

FIG. 15 is a plot of a fatigue strength curve of a flexible material, according to aspects of the present disclosure.

FIG. 16 is a table showing force, stress, and energy results of a fluid chamber compression simulation, according to aspects of the present disclosure.

FIG. 17 is a cross-sectional view of a fluid chamber assembly being compressed by a roller, according to an embodiment of the present disclosure.

FIG. 18 is a cross-sectional view of a fluid chamber assembly being compressed by a roller, according to an embodiment of the present disclosure.

FIG. 19 is a cross-sectional view of a membrane of a fluid chamber assembly deforming as a result of fluid pressure within the fluid chamber, according to aspects of the present disclosure.

FIG. 20 is a cross-sectional view of a fluid chamber assembly that includes a flexible membrane having a negative camber, according to one embodiment of the present disclosure.

FIG. 21 is a flow diagram illustrating a method of assembling fluid chamber, according to one aspect of the present disclosure.

FIG. 22 is a flow diagram illustrating a method for pumping fluid from a patient's eye, according to an embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the therapeutic devices are described in terms of eye-mountable devices configured to pump fluid (e.g., aqueous humor) from a human eye, it is understood that it is not intended to be limited to this application. The devices and systems are equally well suited to any application requiring pumping of fluids. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Presently, treatment of glaucoma mainly consists of periodically administering pharmaceutical agents to the eye to decrease IOP. These drugs can be delivered by, for example, injection or eye drops. However, the effectiveness of pharmaceuticals can greatly vary from patient-to-patient. Furthermore, effective treatment of glaucoma requires adherence to rigid dosage schedules that can be difficult to follow for some patients.

Another way to reduce IOP involves removing quantities of fluid from inside the patient's eye. However, current devices are not suitable or practical for therapeutic use. For example, devices to remove fluid from the eye need to be small enough to be implanted into the patient at a practical location, such as the patient's eye cavity. Due to the invasiveness of implanting such a device, the device should be able to operate independently for a period of time. Thus the device must be able to operate efficiently in a restricted space, and must be reliable enough to require little or no maintenance. The present disclosure proposes implantable peristaltic micropump assemblies for pumping fluid from inside a patient's eye.

A peristaltic pump acts by radially compressing a tube with one or more rotating rollers. This permits a fluid to be pumped without the fluid contacting any portion of the pump mechanism, other than the tube itself. The tube is disposable, such that when the fluid begins to undergo a physical change (e.g., coagulation) or a chemical change (e.g., oxidation), or when a different fluid is desired to be pumped, a fresh tube may be inserted into the pump to prevent contamination. Often these tubes are made of silicone, although other materials may be used.

Traditional peristaltic pumps suffer from fatigue and lifetime problems. In order to prevent backward leakage of fluids that would decrease the pump's efficiency, it is desirable to crush the tube completely, so that its inner walls touch and gaps between the walls are minimized or eliminated. This creates substantial stress on the edges of the tube, such that after repeated cycling the tube material experiences fatigue-related failures. Such failures are typically prevented by replacing the tube before fatigue sets in.

Traditional peristaltic pumps also require substantial power to operate, as the majority of the energy consumed by the pump is expended crushing the tube (deformation energy), and only a fraction goes toward moving the fluid forward. For peristaltic pumps powered by rechargeable batteries, this leads to short battery life and frequent recharging. With the addition of an induction coil, batteries can be charged by wireless induction, such that there is no need to connect a physical charging cable.

Energy consumption of a peristaltic pump can be reduced by reducing the thickness of the tube walls. However, this increases stress on the tube walls and therefore decreases the lifespan of the tube. Furthermore, because flexible plastic or rubber tubing is formed by extrusion of a cylindrical member with a cylindrical hole along its longitudinal axis, there is a practical limit to how thin tube walls can be made for micropump applications. These and other limitations have prevented traditional peristaltic pumps from being used in implantable medical devices, as the replacement of tubes would require surgical removal and replacement of the device, and even with inductive charging a short battery lifetime makes the devices prohibitively inconvenient to use in vivo.

The present disclosure describes micropump assemblies that overcome the challenges described above. In that regard, the micropump assemblies described herein provide advantageous arrangements of components and features that allow the micropumps to reliably and efficiently pump fluid from a patient's eye while maximizing the lifetime of the device.

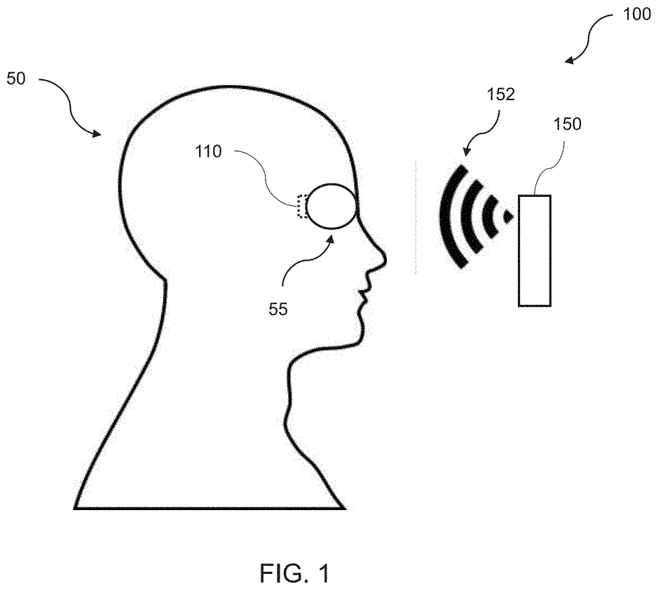

FIG. 1 is a diagrammatic view of a micropump system 100, according to one embodiment. The system 100 includes an eye-mountable micropump 110 coupled to an eye 55 of a patient 50, and a wireless transmission device 150 configured to wirelessly transmit electrical power 152 and/or electrical signals to the micropump 110. The micropump 110 is sized and shaped to be permanently or semi-permanently attached to the patient's eye 55. In particular, the micropump 110 is configured to be positioned within an ocular cavity proximate the eye 55. In some embodiments, the micropump 110 can be positioned at different locations with respect to the patient's eye, such as below the eye 55, above the eye 55, inside the eye 55, or inside any suitable anatomical structure that allows the micropump to pump fluid from the eye 55.

Because the micropump 110 may not be easily accessible for charging or reprogramming, the micropump 110 is configured to wirelessly receive electrical power 152 and/or electrical signals from the wireless transmission device 150. The wireless transmission device 150 includes circuitry and components to send electrical power, such as coils, transformers, power supplies, batteries, or other circuitry. Additionally, the wireless transmission device 150 can include wireless communication components to transmit and/or receive data in the form of wireless signals to/from the micropump 110. As explained further below, the micropump 110 can also include wireless electronic components for receiving electrical power and/or electrical signals form the transmission device 150. The micropump 110 can include a battery and a processing component that allow it to operate independently for a period of time (e.g., days, weeks, months) without receiving power or signals from the transmission device 150.

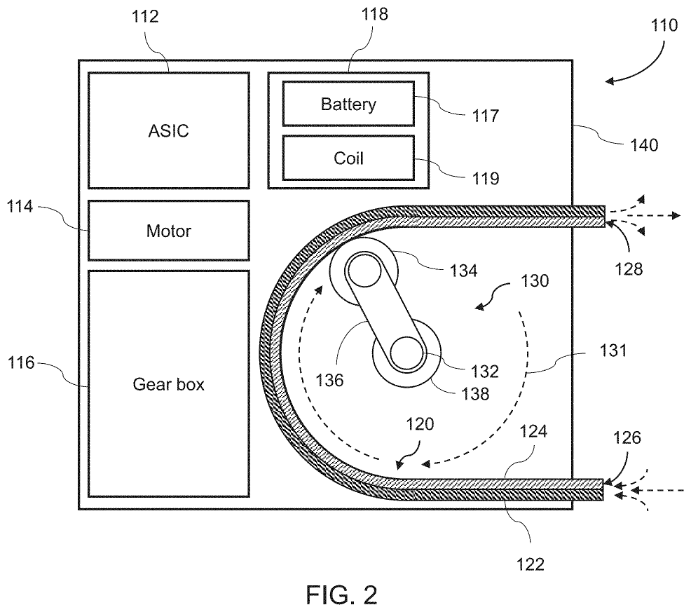

FIG. 2 is a diagrammatic schematic view of a micropump assembly 110, according to one embodiment of the present disclosure. The micropump assembly 110 includes a compressible fluid chamber 120 and a driver assembly 130 configured to compress the fluid chamber 120 to move fluid through the fluid chamber 120. The driver assembly 130 is actuated and controlled by a plurality of electronic and mechanical components, such as an application-specific integrated circuit (ASIC) 112, an actuator or motor 114, a gear box 116, and a power circuit 118. The power circuit 118 includes a battery 117, and a coil 119 configured to receive electrical power from a wireless source, such as the wireless transmission device 150 shown in FIG. 1. The power circuit 118 is configured to supply electrical power to the components of the micropump 110, including the ASIC 112, and the motor 114. The power circuit 118 separately provides electrical power to the ASIC 112 and the motor 114, in some embodiments. In other embodiments, the power circuit 118 provides electrical power to the ASIC 112, which distributes the electrical power to the other components of the micropump assembly 110, including the motor 114.

The ASIC 112 is configured to control an output of the motor 114, thereby controlling the performance (e.g., flow rate) of the micropump 110 assembly. The ASIC 112 operates according to a protocol, which comprises computer code instructions saved in a memory device of the ASIC 112. The protocol is defined by one or more parameters, such as time, number of cycles, physiological measurements, battery life, etc. Thus, the ASIC 112 is configured to control operation of the micropump assembly 110 while the assembly 110 is implanted in the patient. It will be understood that, although the ASIC 112 is shown as a single component in FIG. 2, the micropump assembly 110 may comprise a plurality of individual integrated circuits or other circuitry that is configured to carry out the functions of the assembly 110.

The power circuit 118 and/or the ASIC 112 provide electrical power to the motor 114, which is configured to activate the driver assembly 130 via the gear box 116. The gear box 116 is configured to modify or convert a torque provided by the motor 114, and apply the modified torque to the driver assembly 130. In that regard, the gear box 116 comprises one or more gears or stages of gears to increase or decrease the torque applied by the motor 114. Thus, the gear box 116 can also be appropriate referred to as a torque converter. In an exemplary embodiment, the gear box 116 is configured to increase the torque applied by the motor 114. The increased torque provided by the gear box 116 can help to overcome friction on the driver assembly 130 caused by, e.g., the roller 134 on the compressible fluid chamber 120.

In an exemplary embodiment, the motor 114 is an electrostatic motor, such as the Silmach PowerMEMS® electrostatic motor. However, other motors are also contemplated by the present disclosure, including lavet-type motors, piezoelectric motors, step motors, brushless motors, or any other suitable type of motor.

The driver assembly 130 includes a drive shaft 132 configured to rotate about a first axis and a compressing member or roller 134 rotatably coupled to the drive shaft 132 by a rotor 136. The rotor 136, which can also be referred to as a crank, couples the roller 134 to the drive shaft 132 such that the roller 134 travels about the first axis of the drive shaft 132 along a circumference 131 or circular path when the drive shaft 132 is rotated by the motor 114 via the gear box 116. The roller 134 is rotatably coupled to the rotor 136, such that the roller can rotate about a second axis while traveling along the circumference 131. As described further below, the drive shaft 132 and roller 134 can each comprise one or more ball bearings, such as the drive shaft bearing 137, to reduce friction, and therefore reduce the amount of torque required to rotate the driver assembly 130.

As the driver assembly 130 rotates the roller 134 along the circumference, the roller compresses the fluid chamber 120 in a circular motion around the circumference 131. This circular compression causes the peristaltic pumping action that moves fluid into the fluid chamber 120 through an inlet 126, through the fluid chamber 120 in the circumferential direction 131, and out the fluid chamber 120 through an outlet 128. As an example, when the micropump assembly 110 is implanted onto the patient's eye 55, the inlet 126 can be coupled the eye 55 to receive the aqueous humor, and the outlet 128 can be positioned outside the eye 55, for example, in the ocular cavity. When the micropump assembly 110 is activated, the micropump 110 draws the fluid from inside the eye 55, and expels the fluid outside of the eye 55, thereby reducing the patient's intraocular pressure (IOP).

The fluid chamber 120 can include a round outer portion, or ring 122, and a flexible membrane 124 coupled to the hard outer ring 122 and opposing an inner surface of the outer ring 122. The outer ring 122 can comprise a material that is relatively harder and/or more rigid than the flexible membrane, such as a plastic. As will be explained further below, compression of the fluid chamber 120 involves deforming the membrane 124 toward the outer ring 122 to close or restrict a channel formed between the outer ring 122 and the membrane 124. As will be understood with reference to the embodiment of FIG. 2, the outer ring 122 is not necessarily circular. For example, in FIG. 2, the outer ring 122 includes a circular arc portion and a linear portion. In that regard, the outer ring 122 is not closed, but forms a U-shape. Thus, although the term “ring” is used with respect to the outer portion or ring 122, this is in no way limiting to closed, circular shapes.

The components of the micropump assembly 110, including the driver assembly 130, fluid chamber 120, ASIC 112, motor 114, gear box 116, and power supply circuit 118 are coupled to and/or contained within a housing 140. The housing 140 is sized and shaped to be implanted into an ocular cavity of the patient 50. The housing 140 is configured to contain and protect the components of the micropump assembly 110 from physical and/or chemical damage. In some embodiments, the housing 140 provides a waterproof casing for one or more electrical components of the device, such as the ASIC 112, the power circuit 118, and the motor 114. The housing 140 may also be configured to protect one or more components from chemical damage. In some embodiments, the housing 140 is configured to protect the mechanical components, such as the gear box 116 and the driver assembly 130 from foreign material that could interfere with or inhibit the mechanical performance of the micropump 110.

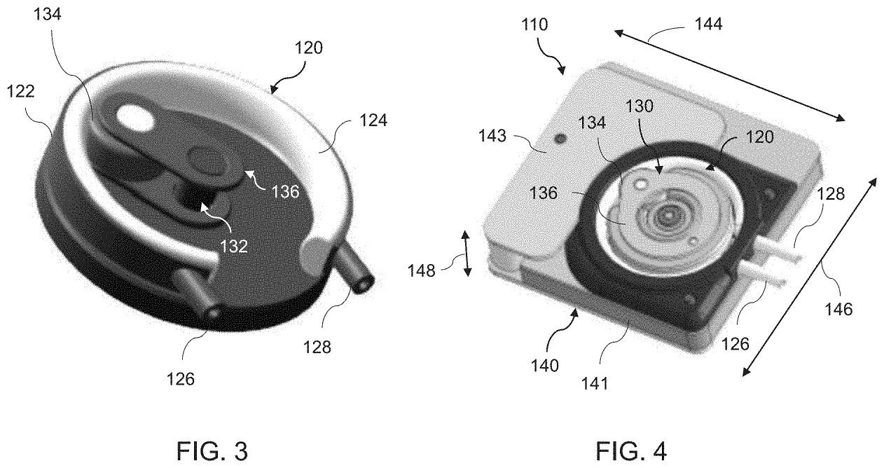

FIG. 3 is a perspective view of a drive assembly and fluid chamber 120 of the micropump assembly 110, according to one embodiment. As in the embodiment shown in FIG. 2, the embodiment of FIG. 3 includes a drive shaft 132 and a roller 134 rotatably coupled to the drive shaft 132 by the rotor or crank 136. The roller 134 is configured to travel in a circular motion about a first axis of the drive shaft 132. The fluid chamber 120 includes a hard outer ring 122, and a flexible membrane 124 opposing an inner face or surface of the outer ring 122. An inlet 126 and an outlet 128 of the fluid chamber 120 are integrally formed with the outer ring 122 and are configured to direct ingress and egress of fluid through the fluid chamber 120. However, in other embodiments, the inlet 126 and/or outlet 128 are not integrally formed with the outer ring 122. For example, the inlet 126 and/or outlet 128 can be formed of the membrane 124, or formed of both the membrane 124 and the outer ring 122. In other embodiments the inlet 126 and/or outlet 128 can comprise physically separate components that are attached to the outer ring 122 and/or the membrane 124. As described above, as the roller 134 rotates about the circumference 131, the membrane 124 is deformed or pressed against the outer ring 122 to move fluid through the fluid chamber 120 in a peristaltic motion toward the outlet 128. To reduce friction, the roller 134 is also configured to rotate or spin in a planetary motion about a second axis and around the first axis. Further, in some embodiments,

FIG. 4 is a perspective view of a micropump assembly 110, according to an embodiment of the present disclosure. Similar to the assembly 110 shown in the FIG. 2, FIG. 4 shows a driver assembly 130 and a fluid chamber 120 contained within a housing 140. In contrast to the embodiments shown in FIGS. 2 and 3, the rotor or crank 136 shown in FIG. 4 has a circular shape and is positioned around the drive shaft 132. The circular rotor 136 couples the roller 134 to the drive shaft 132 such that the roller 134 travels around the first axis along a circumference.

The assembly 110 includes a housing 140 that houses the components of the assembly 110, including the driver assembly 130 and the fluid chamber 120. Other components are also positioned within the housing, such as the ASIC 112, motor 114, gear box 116, power circuit 118, or any other suitable components. The housing 140 shown in FIG. 4 includes multiple pieces, including a first piece 141 and a second piece 143. The second piece 143 may act as a cover for one or more components such as the gear box 116 and the motor 114. The housing 140 is configured to contain the components of the assembly 110 within a space small enough to be implanted into the patient. In that regard, the assembly 110 comprises a length 144, a width 146, and a height 148. In an exemplary embodiment, the length 144 is about 9 mm, the width 146 is about 9 mm, and the height 148 is about 2 mm. However, the dimensions can be modified as appropriate for the application. For example, the length 144, width 146, and/or height 148 can range from less than 1 mm to more than 30 mm.

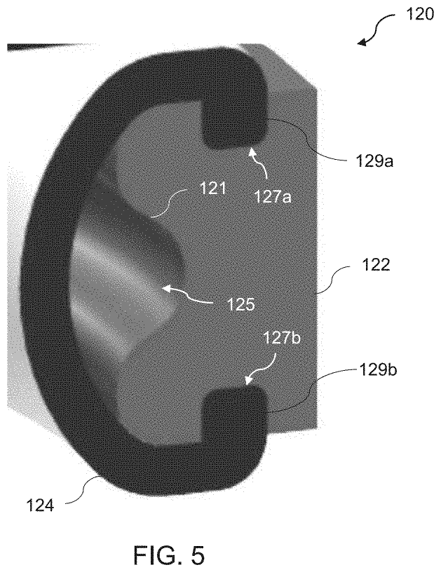

FIG. 5 is a perspective cross-sectional view of the fluid chamber 120 of the assembly 110. The fluid chamber 120 includes an outer ring 122, and a flexible membrane 124 coupled to the outer ring 122 to define a fluid channel 125. The flexible membrane 124 comprises an elastomeric material such as silicone, while the outer ring 122 comprises a relatively harder material, such as a plastic. The membrane 124 is positioned over, or opposing, an inner surface 121 of the outer ring 122. The inner surface 121 comprises a valley that partially defines the fluid channel 125. The membrane 124 is attached to the outer ring 122 at a first groove 127a on a top side of the outer ring 122, and a second groove 127b on an opposing bottom side of the outer ring 122. A first ridge 129a of the membrane 124 is positioned within the first groove 127a, and a second ridge 129b of the membrane 124 is positioned within the second groove 127b. The first and second ridges 129a, 129b can be attached to the outer ring 122 by any suitable method, including a weld, thermal bond, adhesive, or a mechanical fit (e.g., interference fit). It will be understood that, in some embodiments, the first and second ridges 129a, 129b, are formed of opposing edges of a rectangular membrane.

As explained above, the outer ring 122 may comprise a material that is relatively harder and/or more rigid than the membrane 124. Accordingly, while the membrane 124 is configured to be deformed by the roller 134, the outer ring 122 may be configured to retain its shape, even with applied pressure from the roller 134. In a relaxed or undeformed state, the membrane 124 spans across the curved inner surface 121 of the outer ring 122 such that a space exists in the fluid channel 125 for a fluid to pass through. When the roller 134 passes over the membrane 124, the membrane 124 is deformed toward the inner surface 121 of the outer ring 122 to reduce or close the space in the fluid channel 125. The membrane 124 is thus deformed in a circular fashion around the circumference to create a peristaltic pumping action that moves a fluid through fluid chamber 120 toward the outlet 128.

The fluid chamber 120 described above exhibits certain advantages to existing fluid chambers. For example, the coupling of the membrane 124 to the hard outer ring 122 can reduce the stress applied to the fluid chamber 120 when compressed by the driver assembly 130. In that regard, as opposed to flexible tubes that are compressed by collapsing one side of the tube toward the other side of the tube, compressing the fluid chamber 120 shown in FIG. 5 is accomplished by deforming the flexible membrane against the relatively hard or rigid outer ring 122. Thus, when the membrane 124 is relaxed, the channel 125 of the fluid chamber 120 between the membrane and the outer ring 122 is relatively unrestricted. Compressing the membrane 124 against the outer ring 122 can be achieved with relatively little stress to any given portion of the flexible membrane 124. Furthermore, because the outer ring 122 provides the structural integrity to define the channel 125, the flexible membrane can be formed of a soft elastomeric material that can be more easily compressed. Furthermore, the smooth, round surface 121 can also reduce the amount of stress on the membrane 124 during compression. Thus, the fluid chamber 120 can be compressed with less resistance than what would be required with flexible tubing. Furthermore, because the membrane 124 undergoes relatively little stress, the durability and lifespan of the fluid chamber 120 can be increased.

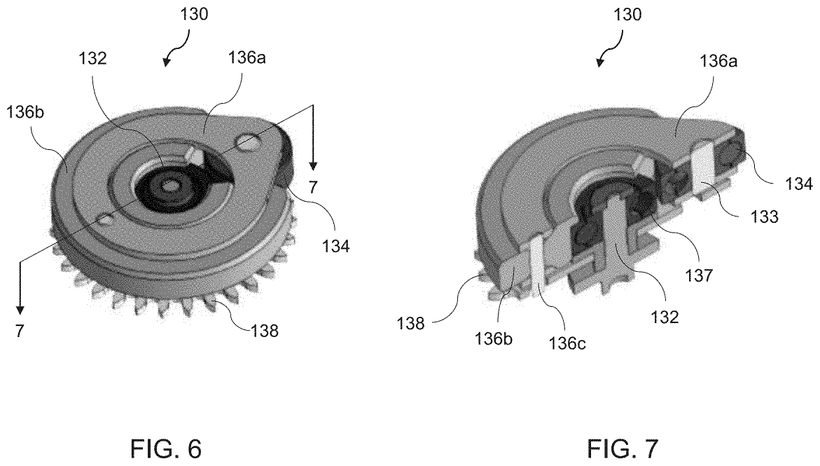

FIGS. 6 and 7 depict a driver assembly 130 of the micropump assembly 110 shown in FIG. 4, according to one embodiment of the present disclosure. In particular, FIG. 6 is a perspective view of the driver assembly 130, and FIG. 7 is a perspective cross-sectional view of the driver assembly 130 taken along the line 7-7. As in FIG. 4, the driver assembly 130 includes a drive shaft 132 and a rotor or crank 136, which comprises a top plate 136a and a bottom plate 136b. The driver assembly 130 also includes a gear 138 fixedly coupled to the top plate 136a and bottom plate 136b of the rotor by a rotor pin 136c. The gear 138 is positioned concentrically with the drive shaft 132 and the first axis. The pin 136c couples the gear to the rotor such that torque applied to the gear 138 rotates the rotor 136, and therefore the roller 134. The drive shaft 132 is concentrically coupled to a first bearing 137 to rotate about a first axis. Similarly, the roller 134 comprises a bearing concentrically coupled to a roller bearing pin 133 to rotate about a second axis.

Because it is desired that the entire micropump assembly 110 is sized and shaped to be implanted into a patient (e.g., inside the ocular cavity), the components of the driver assembly 130 can be low-profile. For example, in some embodiments, the ball bearings of the drive shaft 132 and the roller 134 have a diameter of 2 mm or less.

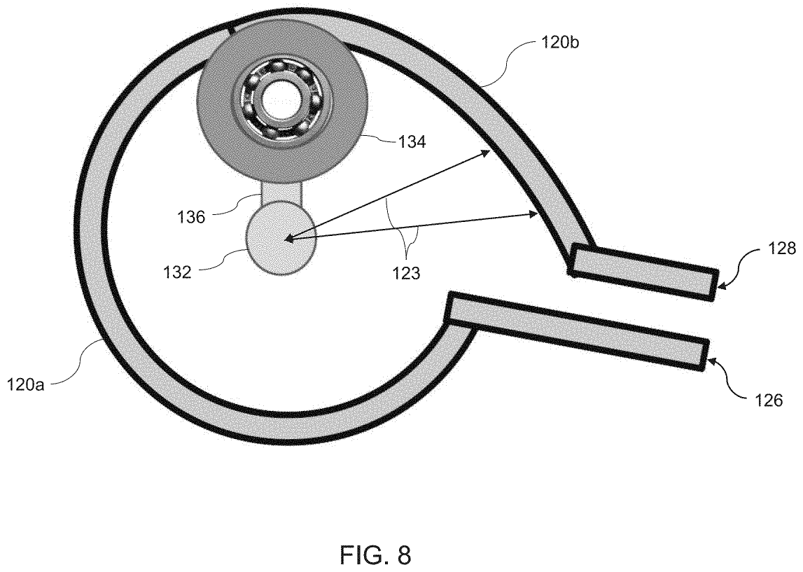

FIG. 8 is a top view of a driver assembly and a fluid chamber 120, according to one embodiment of the present disclosure. The driver assembly 130 of FIG. 8 may include similar or identical components as the assembly 130 shown in FIGS. 2 and 3, such as a drive shaft 132, a crank 136, and a roller 134. The fluid chamber 120 includes a circular portion 120a and a non-circular portion or spiral portion 120b. In that regard, the non-circular portion 120b is shaped and arranged such that a radius 123 between the drive shaft 132 and the fluid chamber increases in a clockwise direction of the fluid chamber 120. Thus, with the configuration shown in FIG. 8, the micropump assembly 110 can function as a pump over the circular portion 120a, and as a flow controller for the rest of the cycle over the non-circular portion 120b. In that regard, as the roller 134 passes over the circular portion 120a, the fluid chamber 120 is fully compressed, but when the roller 134 passes over the non-circular portion 120b, the fluid chamber 120 is only partially compressed, thereby reducing the hydraulic resistance as the roller 134 rotates clockwise over the non-circular portion 120b. When a positive pressure gradient exists across the micropump 110 (e.g., when the IOP is relatively high), fluid may flow from the inlet 126 to the outlet 128 even without pumping. In this case, pumping is mainly used for clearing and preventing clogs. When a stepper motor is used as the actuator or motor 114, the motor 114 can be controlled to stop at any desired angular location. Thus, the stepper motor 114 can control the roller 134 to stop at a desired position along the non-circular portion 120b. Because the compression of the fluid chamber 120 by the roller 134 gradually decreases as the roller 134 moves clockwise along the non-circular portion 120b, the micropump 110 can act as a variable flow controller to adjust the flow of fluid through the micropump 110 that is caused by the positive pressure gradient. For example, if the motor 114 stops the roller 134 over the circular portion 120a, the fluid chamber 120 is fully compressed such that flow through the fluid chamber 120 is effectively zero. By contrast, when the roller 134 is moved to a location along the non-circular portion 120b that is near the outlet 128, the fluid chamber 120 may not be compressed at all, or only minimally compressed, such that fluid flow through the chamber 120 is effectively unrestricted. The motor 114 can also control the roller 134 to stop at a desired location along the non-circular portion 120b corresponding to a desired amount of compression of the fluid chamber 120, and therefore adjusting the flow of fluid through the chamber 120 to a desired amount.

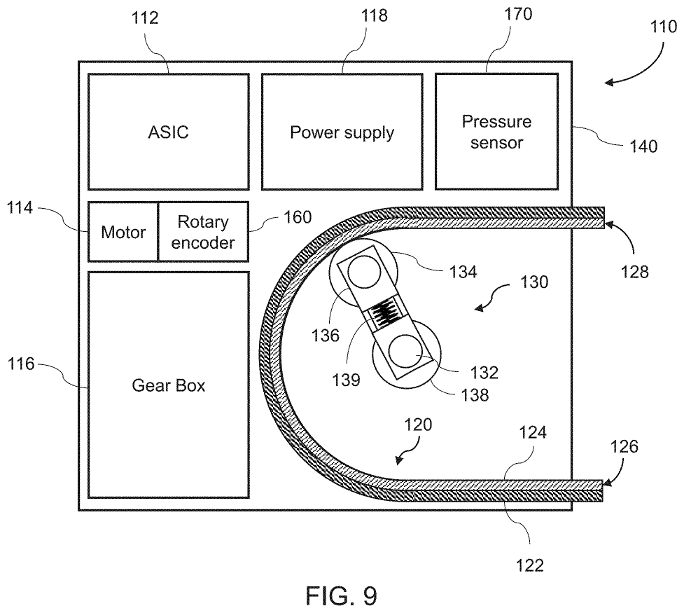

FIG. 9 is a diagrammatic schematic view of a micropump assembly 110, according to another embodiment of the present disclosure. The micropump assembly 110 embodiment shown in FIG. 9 can include similar or identical components as the embodiment shown in FIG. 2. For example, the embodiment shown in FIG. 9 includes an ASIC 112, a motor 114, a gear box 116, a power circuit 118, a fluid chamber 120, and a driver assembly 130. Additionally, the micropump assembly 110 includes a rotary encoder 160 in communication with the motor 114, a pressure sensor 170, and a rotor spring 139. The rotary encoder 160 is communicatively coupled to the motor 114 and configured to provide an indication or feedback to indicate the rotational position of the motor 114 to the ASIC 112 and/or motor 114. The rotary encoder 160 can be used to control pumping of fluid through the micropump 110 with volumetric precision. For example, in some embodiments, the micropump assembly 110 can be used to deliver pharmaceutical agents to the patient. The rotary encoder 160 can be used to provide feedback to the ASIC 112 to control dosing of the pharmaceutical with nanoliter precision.

The pressure sensor 170 measures a pressure or pressure gradient across the fluid chamber 120. The pressure sensor 170 is communicatively coupled to the inlet 126 of the fluid chamber 120 to measure a fluid pressure from a source, such as the IOP of the patient's eye 55. The pressure sensor 170 provides signals to the ASIC 112 representative of a measured fluid pressure. The ASIC 112 adjusts performance of the micropump 110 based on the feedback provided by the pressure sensor 170. For example, as IOP fluctuates throughout the day, the ASIC 112 may control the micropump 110 to pump relatively greater volumes of fluid during portions of the day when the IOP measured by the pressure sensor 170 is relatively high. By contrast, the ASIC 112 may control the micropump 110 to pump relatively smaller volumes of fluid, or cease pumping altogether, during portions of the day when the IOP measured by the pressure sensor 170 is relatively low. In this manner the pressure sensor 170 and the ASIC 112 function as a pressure controller. For example, the ASIC 112 can be programmed to maintain the IOP, as measured by the pressure sensor 170, at a desired pressure.

The driver assembly 130 includes a rotor spring 139 positioned between the drive shaft 132 and the roller 134. The spring 139 can be biased to push the roller 134 toward the fluid chamber 120. In that regard, the spring 139 can regulate the force applied by the roller 134 on the membrane 124 of the fluid chamber 120. The spring 139 of the rotor 136 may also exhibit a particular amount of travel, thereby adjusting the radius or distance between the roller and the drive shaft 132. The spring 139 can comprise one or more of a variety of mechanisms to impart a spring force, including compression springs, membranes, magnets, leaf springs, torsion springs, coil springs, or any other suitable type of spring.

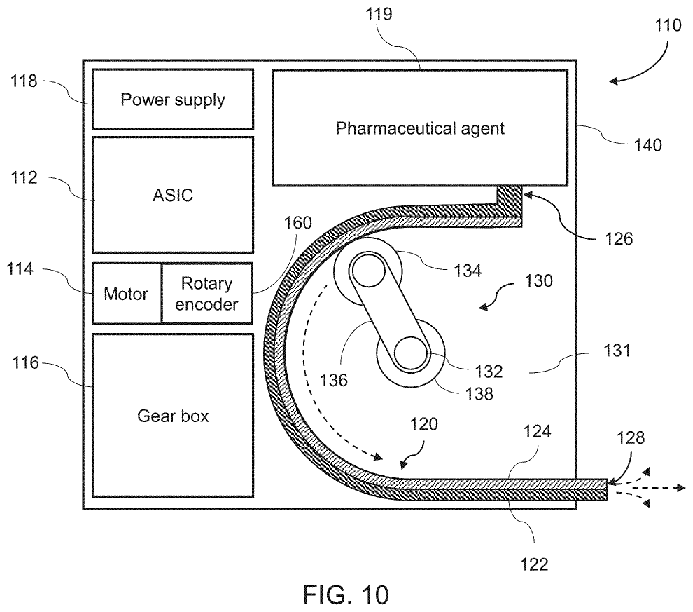

FIG. 10 depicts another embodiment of the micropump assembly 110 that is used for delivering a pharmaceutical agents to the patient. The micropump assembly 110 includes a reservoir 119 containing the pharmaceutical agent, with the reservoir 119 in communication with the inlet 126 of the fluid chamber 120. It will be understood that the driver assembly 130 of the embodiment in FIG. 10 is shown rotating in a counter-clockwise fashion toward the outlet 128. The outlet can be connected to or otherwise in fluid communication with an anatomical structure of the patient, such as an organ (e.g., the eye) or a tissue. The micropump assembly 110 shown in FIG. 10 includes a rotary encoder 160 in communication with the ASIC 112 and the motor 114. The rotary encoder 160 can be used as described above to precisely control the volumetric flow of the pharmaceutical agent into the patient via the outlet 128. In some embodiments, the motor 114, rotary encoder 160, and ASIC 112 are configured to enable microdosing of the pharmaceutical agent with nanoliter precision.

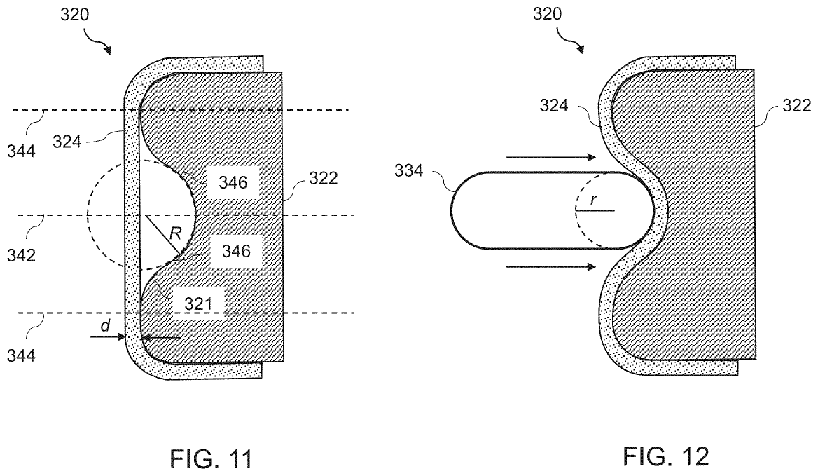

As mentioned above, embodiments of the present disclosure include fluid chambers having a two-part construction with a bell-shaped fluid channel instead of an extruded flexible tube with a circular cross-section. FIGS. 11 and 12 show diagrammatic cross-sectional views of a fluid chamber 320 including a bell-shaped fluid channel, according to aspects of the present disclosure. The cross-sectional views shown in FIGS. 11 and 12 are exemplary of the cross section of the fluid chamber 120 shown in FIG. 2, with membrane 324 as an exemplary embodiment of the membrane 124, and the hard outer ring 322 as an exemplary embodiment of the hard outer ring 122. The fluid chamber 320 includes a hard outer portion or ring 322 having a bell-shaped groove 321 or channel on its inner surface, with a flexible membrane 324 (e.g., a TPE or silicone membrane) sealed across its top by means of adhesives or welding (e.g., laser, ultrasonic, or thermal welding), forming an enclosed channel with a roughly D-shaped or bell-shaped cross section. The membrane 324 may have a C-shaped cross section that extends over the edges of the hard plastic outer ring, and may be held in place by an adhesive to hard outer ring 322, although this is not required, as laser welding may produce very thin, strong weld lines that seal the membrane 324 across the trough or channel of the hard outer ring 322, forming the bell-shaped channel or fluid chamber. Both the hard outer ring 322 and the flexible membrane 324 may be fabricated by injection molding, although the membrane 324 may more easily be fabricated by extrusion.

The curved inner surface 321 between outer inflection points 344 can be defined by one or more types of curves. For example, in the embodiment of FIG. 11, a portion of the curved inner surface 321 is defined by a circle of radius R. The curved surface 321 changes from a circular profile to an inflected arcuate profile at inner inflection points 346. The curved surface 321 is centered and symmetrical about a center line or plane 342. As explained further below, in one embodiment, the radius R of the curved surface 321 can be equal to or approximately equal to the radius r of the roller fillet 334 plus the thickness d of the membrane 324. In some embodiments, the curved surface 321 between outer inflection points 344 can be defined by other types of curves, such as a sinusoidal, Gaussian, Lorentzian, Voigt, symmetrical spline, mirrored biarc, etc. For example, in one embodiment, at least a portion of the curved surface 321 between outer inflection points 344 can be represented by a Gaussian function of the form:

$$f ( x ) = a {e}^{\frac{- {x}^{2}}{2 {b}^{2}}}$$

Where a is related to the height of the curve's peak and b is related to the width of the bell-shape. In other embodiments, at least a portion of the curved surface 321 can be represented by a sinusoidal function, wherein the outer inflection points are aligned with consecutive troughs of the sin wave. In some aspects, one or more of the inflection points can be positioned between a convex portion of the curved surface 321 and a concave portion of the curved surface 321. In other embodiments, at least a portion of the curved surface 321 can be defined by one or more of: a parabola, hyperbola, ellipse, spiral, a polynomial curve, exponential curve, sigmoid, or any other suitable type of curve.

Similarly, the cross-sectional shape or profile of the roller fillet 334 can be made to be geometrically compatible with the curved surface 321. For example, in some embodiments, the roller fillet 334 and the curved surface 321 are defined by the same type of curve such that the roller fillet 334 can more evenly distribute force on the membrane 324 to deform against the curved surface 321 of the hard outer ring 322.

Referring to FIG. 12, in operation, the membrane 324 is compressed by a roller mechanism 334 to contact the bell-shaped groove 321 on the inner surface of the hard outer ring 322. According to at least one embodiment of the present disclosure, the roller 334 is a wheel bearing with a plastic fillet, over-molding, or cover, and power is transferred from the motor to the roller 334 with minimal friction by means of a motor gear to which the bearing is attached via a pin although other mechanical or electromechanical transmission mechanisms may be employed to achieve the desired result. The roller 334 or roller fillet comprises a rounded cross section with a curvature characterized by the fillet radius r. The hard outer ring 322 may be annular or substantially circular in shape or may have other shapes, such as a spiral or hybrid of spiral and circular.

The two-part construction of the fluid chamber 320 can decrease the maximum stress experienced by the membrane 324 during compression. For example, the membrane 324 may experience significantly less stress during compression than conventional flexible tubing. The reduction in maximum membrane stress has a nonlinear beneficial effect on the endurance or cycle lifetime of the membrane 324, and therefore on the lifetime of the peristaltic pump with D-shaped or bell-shaped channel.

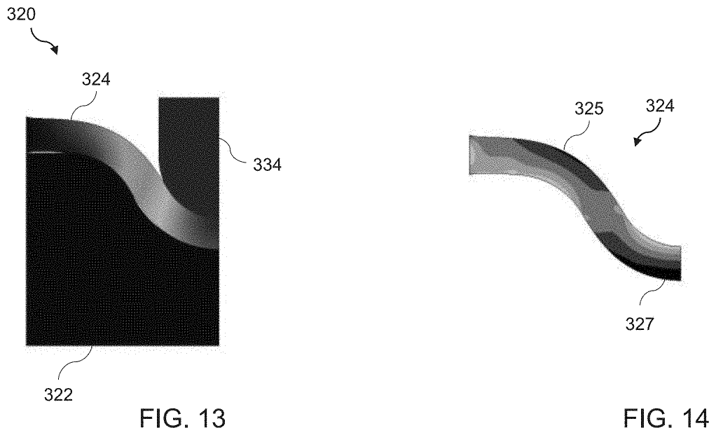

Flexible materials such as silicone rubber exhibit a “fatigue limit”, wherein repeated stresses above this limit lead to substantially reduced endurance (measured in flexure cycles), whereas repeated stresses below this limit degrade the material much less, and the material is thus able to survive many more cycles. In that regard, FIGS. 13 and 14 are diagrammatic graphical views of the displacement (in um) and stress (in MPa) experienced by the flexible membrane 324 during compression by a roller 334. It will be understood that FIGS. 13 and 14 show only half of the cross-section of the fluid chamber 320 during compression.

In FIG. 13, a graphical view of the displacement of the membrane 324 is shown while the membrane 324 is fully compressed by the roller 334. The displacement is highest at the bottom of the bell-shaped groove of the hard outer ring 322, and lowest at the top of the bell-shaped groove, which is near the location at which the membrane 324 is attached to the hard outer ring 322. FIG. 14 shows a graphical map of the stress experienced by the membrane 324 during maximum compression. The stress may be highest at the regions of greatest curvature, including near the top or shoulder 325 of the bell-shaped curve, and at the bottom 327 of the bell-shaped curve. In this configuration, the maximum stress experienced by the membrane 324 during compression is approximately 0.7 MPa. However, the maximum stress may be higher or lower is some configurations, such as when the thickness of the membrane 324 increases, or when the radius R of the bell-shaped groove decreases.

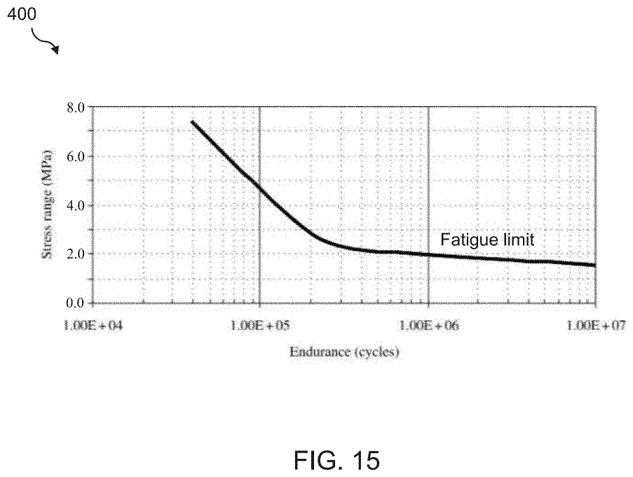

The lifespan of the membrane 324, measured in cycles, is a function of the maximum stress experienced by the membrane 324. FIG. 15 shows a representative rubber fatigue strength curve 400 for an example material, though not necessarily the exact curve for a given material used in the foregoing analysis. The fatigue limit of the material can be identified in the plot by the region of the curve with the most gradual slope. By maintaining the maximum stress of a material below this fatigue limit, the number of cycles the material can endure before failure increases exponentially.

As can be seen in the plot 400, the effect of stress on the endurance or cycle life of the example material is small when the stress is substantially above the fatigue limit, such that a stress reduction of 1 MPa may increase the endurance of the example material by only approximately 100 thousand cycles. When the stress on the example material is substantially below the fatigue limit, the effect of stress on the endurance or cycle life of the example material is greatly increased, such that a stress decrease of 1 MPa may increase the endurance of the example material by hundreds of millions of cycles. There is typically a transition region near the fatigue limit, where the sensitivity of the material changes rapidly.

A person of ordinary skill in the art, after becoming familiar with the teachings herein, will recognize that a reduction of membrane stress from, for example, 2.1 MPa for a circular tube to 0.8 MPa for a two-part fluid chamber as described above may be sufficient to maintain the maximum stress below the membrane material's fatigue limit, such that the resulting increase in endurance or cycle life is disproportionate and nonlinear, and such that an endurance in excess of 300 million cycles may be achievable. For an implantable micropump operating at one cycle per second, an endurance on the order of 300 million cycles equates to a life of approximately 10 years, which may not be achievable using a traditional, tube-based peristaltic pump design. Reduced membrane stress also broadens the range of available membrane materials that can be used.

The maximum force on the membrane (and therefore the energy or power requirement for the device) declines as the membrane thickness is decreased. Thus, one may infer that it would be desirable to use a membrane that is as thin as possible in order to increase efficiency of the micropump. However, simulation of an example peristaltic pump with bell-shaped channel for an example glaucoma-mitigating implantable micropump yields unexpected results with regard to maximum stress on the membrane (and therefore its cycle lifetime), wherein a membrane thickness of about 50 um can provide an optimal thickness to maximize longevity of the membrane. As explained further below, the relative pressure (IOP) of the aqueous humor within the eye can be as high as about 9 kPa. This fluid pressure can cause the membrane to bulge outward, imparting stress on the membrane. The stress imparted by the 9 kPa of pressure from the eye on the membrane increases as the thickness of the membrane decreases. At a thickness of 50 um, the membrane 324 experiences an equal amount of stress from full compression by the roller 334, and from the 9 kPa internal pressure of the aqueous humors of the eye. Decreasing membrane thickness below about 50 um shows no additional benefit, as the internal pressure of the aqueous humors of the eye becomes the dominant source of stress, exceeding the amount of stress experienced by the membrane 324 during full compression by the roller 334. Flexible membranes of 50 um thickness can be reliably produced by extrusion.

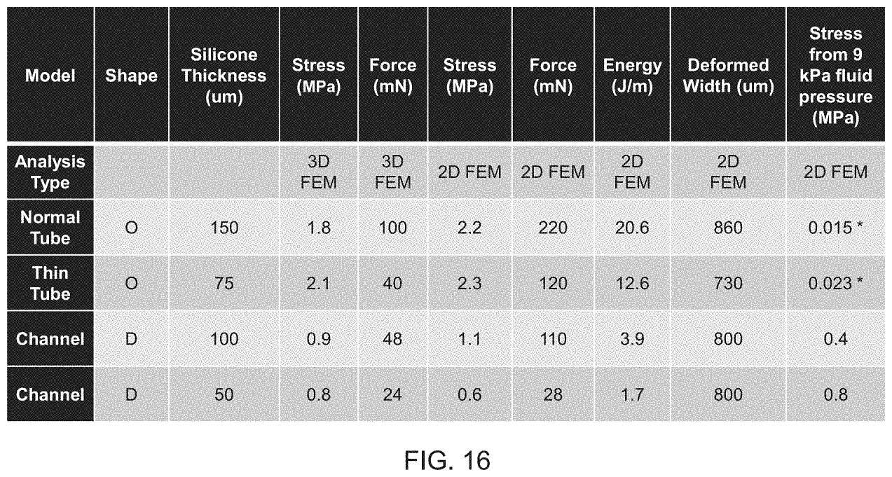

FIG. 16 is a table that shows the results of different simulation parameters for the 2D and 3D simulation of an example peristaltic pump with bell-shaped channel implemented as an example glaucoma-mitigating implantable micropump. The 2D simulation results indicate that for the bell-shaped channel, the maximum stress on the membrane may be reduced by a factor of 2.0-3.8 vs. a traditional peristaltic pump with identical cross-sectional area. 2D results further indicate that the maximum membrane force may be reduced by a factor of 2.0-4.3, and energy or power requirement by a factor of 5.3-7.4, vs. a traditional peristaltic pump. In addition, the tube of a traditional peristaltic pump widens from 770 um to 830 um when fully compressed by the roller, whereas the width of the D-shaped or bell-shaped channel does not change, resulting in a more robust and less mechanically constrained design for the peristaltic pump with bell-shaped channel.

The 3D results from the table of FIG. 16 indicate that the maximum stress on the membrane may be reduced by a factor of 2.0-2.6, and the maximum force may be reduced by a factor of 1.7-2.1 vs. a traditional peristaltic pump. If ratios of energy savings and force reduction are roughly consistent between the 2D and 3D results, then the energy requirement of the peristaltic pump with bell-shaped channel may be reduced by a factor of approximately 4, and likely not less than a factor of 2, vs. a traditional peristaltic pump. After becoming familiar with the teachings herein, a person of ordinary skill in the art will recognize that with traditional, tube-based peristaltic pump designs, it is not possible to adjust design parameters to reduce the maximum tube stress, maximum tube force, and energy requirement simultaneously, while maintaining a consistent flowrate for the pump. The person of ordinary skill in the art will further recognize that the hereinabove demonstrated reductions in membrane stress and power requirement represent a qualitative rather than incremental improvement in the performance of peristaltic pumps for long-life applications without tube replacement.

The peristaltic pump may be sized and/or shaped for a variety of different applications, both inside and outside the human body, and may exhibit a wide range of flow rates and capacities. However, according to at least one embodiment of the present disclosure, the peristaltic pump with bell-shaped channel includes a fluid channel cross-sectional area ranging between 0.03 mm² to 3 mm², and supports a variable flowrate of between zero and about 6 microliters per minute, with a normal operating range of between zero and about 4.2 microliters per minute. In this example, continuous operation of the pump is preferred in order to prevent clogging of the drainage path, although ripples in the flow rate may be considered acceptable.

Furthermore, according to at least one embodiment of the present disclosure, the inlet operating pressure falls within a target range of 5-17 mmHg (0.67-2.27 kPa) with an ideal target of 12 mmHg (1.60 kPa), and a maximum range of 0-80 mmHg (0-10.67 kPa) while the outlet operating pressure falls within a normal expected operating range of 0-20 mmHg (0-2.67 kPa) and a maximum capacity of 70 mmHg (9.33 kPa).

According to at least one embodiment of the present disclosure, the maximum pressure gradient supportable by the peristaltic pump with bell-shaped cavity is −70 to 50 mmHg (−9.33 to 6.67 kPa), and the motor powering the pump mechanism is a MEMS electrostatic stepper motor (e.g., the Silmach PowerMEMS) capable of generating greater than 2.3 uNm of torque at 2.7 RPM or 373 uNm of torque at 1 revolution per hour, and with sufficient power and efficiency to drive the pump mechanism at the hereinabove stated pressures and flowrates without undue power consumption, such that a rechargeable battery of 200 uAh capacity can operate the device for at least one hour of continuous operation. In one example, the flexible membrane is made from biocompatible silicone rubber with a shore hardness of A50, a linear strain-stress curve at functional range, and a Young's modulus of 2 MPa, and the channel or fluid chamber formed between the membrane and the hard plastic ring is equal in cross-sectional area to a cylindrical tube with 300 um inner diameter.

According to at least one embodiment of the present disclosure, the total mechanism of the peristaltic pump with the bell-shaped channel that meets the exemplary criteria listed hereinabove, including a motor, gears, tube chamber, pressure sensor, housing, and wirelessly chargeable battery, is smaller than or equal to about 13 mm×13 mm×2 mm, with a preferred size of 9 mm×9 mm×<2 mm. This is considered acceptable for use as a glaucoma-mitigating micropump that is implantable within the human ocular cavity.

According to at least one embodiment of the present disclosure, the bell-shaped channel is constructed from convex and concave circular curve segments having about the same radius of curvature, as this may simplify manufacturing, and also may also make the properties of the device easier to simulate through finite element modeling or other methods.

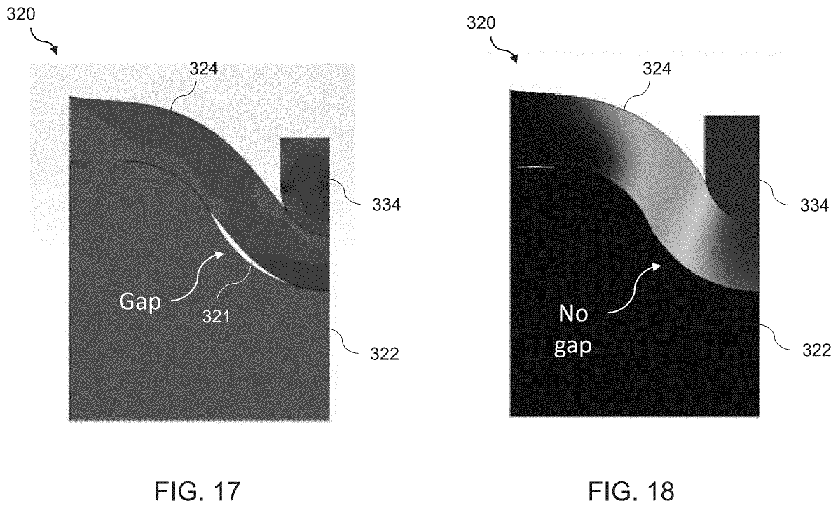

If the roller shape and size is not optimized for the size of the bell-shaped channel and membrane thickness, one or more gaps may form between the flexible membrane and the hard plastic channel when the membrane is maximally compressed by the rotating roller. FIG. 17 is a cross sectional view of a portion of fluid chamber 320 that exhibits a gap between the membrane 324 and the bell-shaped groove 321 of the hard outer ring 322 when the membrane 324 is fully compressed by the roller 334. This gap allows backward-leakage of fluid, reducing both the outlet pressure and the efficiency of the peristaltic pump. In order to prevent such gaps from forming, the roller fillet radius r must increase as a function of increasing channel width. In that regard, FIG. 18 is a cross-sectional view of a portion of a fluid chamber 320 being compressed by a roller fillet 334 having a larger fillet radius than the roller fillet shown in FIG. 17. The radius of the roller fillet 334 is increased to better distribute pressure on the membrane 324 such that it contacts an entire surface of the bell-shaped groove 321. For example, in one embodiment, an optimal roller fillet radius r is approximately equal to the radius of the bell-shaped groove 321 (R, FIG. 11) less the thickness of the membrane 324 (d, FIG. 11). In some embodiments, the optimal roller fillet radius r is slightly larger than the radius R of the bell-shaped groove 321 less the thickness d of the membrane 324. It will be understood, however, that an oversized roller 334 is not desirable, as it increases both maximum stress and maximum force on the membrane material. Beyond a certain size, the roller 334 will no longer fit completely in the channel, which may also create a gap.

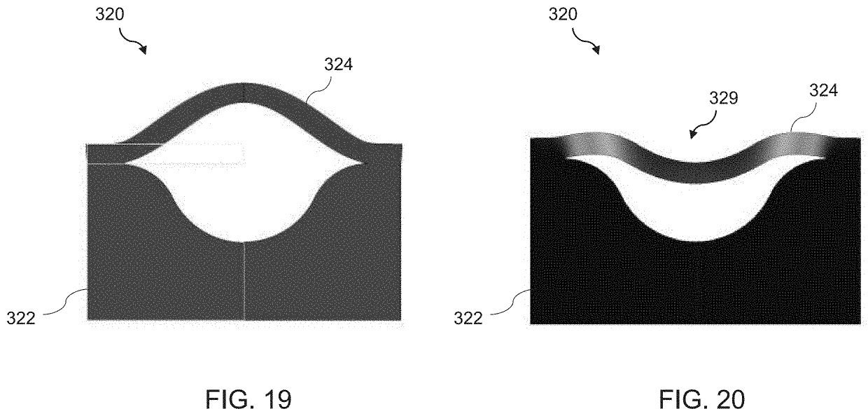

According to at least one embodiment of the present disclosure, an internal pressure within the peristaltic pump with a bell-shaped channel may cause the membrane 324 to bulge upward, as shown in FIG. 19. For example, a 9 kPa internal pressure of the aqueous humor within the human eye may cause a membrane of 50 um thickness to bulge upward by approximately 157 um, causing significant stress on the membrane material. According to at least one embodiment of the present invention, this bulge may be compensated for by manufacturing the membrane 324 with a sag or negative camber 329, as shown in FIG. 20. This sag or negative camber 329 reduces the stress caused by internal pressure, but also reduces the cross-sectional area of the bell-shaped channel between the flexible membrane 324 and the hard outer ring 322, thus reducing the capacity of the pump. A dome-shaped sag reduces the stress even further. A bulge or positive camber may also be designed into the flexible membrane 324 to increase the cross-sectional area of the bell-shaped channel, although this also increases the stress on the membrane material when the membrane 324 is compressed by the roller 334.

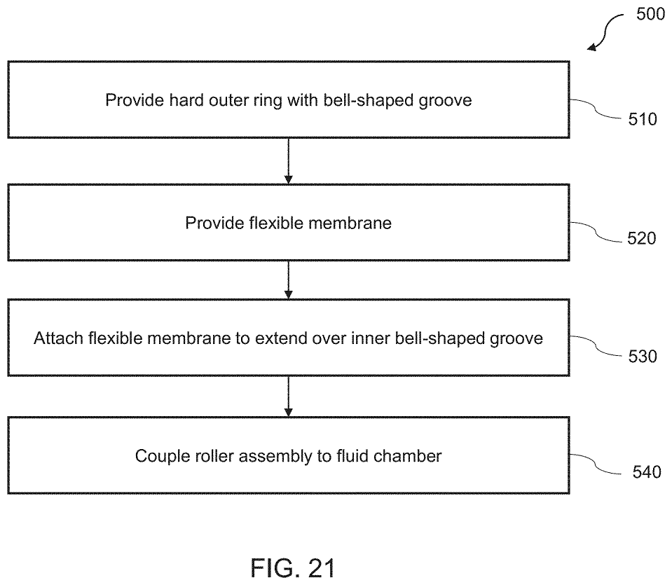

FIG. 21 depicts a method 500 of assembling a peristaltic pump, according to embodiments of the present disclosure. In step 510, a hard outer portion is provided that includes a bell-shaped groove on an inner surface of the hard outer portion. In some embodiments, the hard outer portion comprises a ring. The hard outer ring may comprise a plastic material, in some embodiments. Although referred to as a “ring,” the hard outer ring may not form a circle or closed shape. For example, the hard outer ring can be arranged in a U-shape, spiral shape, polygon, rectangle, or any other suitable shape. In some embodiments, at least a portion of the hard outer ring comprises an arcuate shape or profile, such as a segment of a circle. The hard outer ring may be provided by molding, extruding, machining, or any other suitable process. The bell-shaped groove may be formed during the extrusion or molding of the hard outer ring, or may be formed afterward by machining or any other suitable process.

In step 520, a flexible membrane is provided. The flexible membrane comprises a flexible material such as silicone or TPE, and can be formed by extrusion, molding, or any other suitable process. The flexible membrane is formed to have a thickness appropriate for the application. For example, for a micropump, the thickness of the membrane can be very small (e.g., 25 um, 50 um, 75 um, 100 um, 150 um) in order to reduce the amount of force required to deform the membrane, thereby conserving electrical power. In one embodiment, a 50 um membrane is provided by an extrusion process to produce a flexible sheet of membrane material that can be wrapped around the inner surface of the hard outer ring. The hard outer ring may be flexible in at least one direction, but may be more hard and/or rigid than the membrane such that the hard outer ring experiences no deformation or negligible deformation when the membrane is deformed against the hard outer ring.

In step 530, the flexible membrane is attached to the hard outer ring such that the flexible membrane extends over the inner surface of the hard outer ring. A bell-shaped fluid channel is created or defined by the flexible membrane and the bell-shaped groove of the hard outer ring. The flexible membrane may be attached to the hard outer ring by a laser weld, an adhesive, or any other suitable means of attachment. In one embodiment, the top and bottom surfaces of the hard outer ring comprise grooves inside of which the ends of the flexible membrane are positioned and attached. In other embodiments, the flexible membrane is attached to a flat surface of the hard outer ring, such as the top, bottom, and/or outer surface of the hard outer ring.

In step 540, the fluid chamber formed by the flexible membrane and hard outer ring is coupled to a roller assembly. The roller assembly is coupled to the fluid chamber such that the roller is configured to move across the membrane of the fluid chamber to compress the membrane against the hard outer ring. In some embodiments, the roller assembly is configured to rotate about an axis to move the roller in a circular path. For example, the roller assembly can include a drive shaft and bearing centered around a central axis of the hard outer ring.

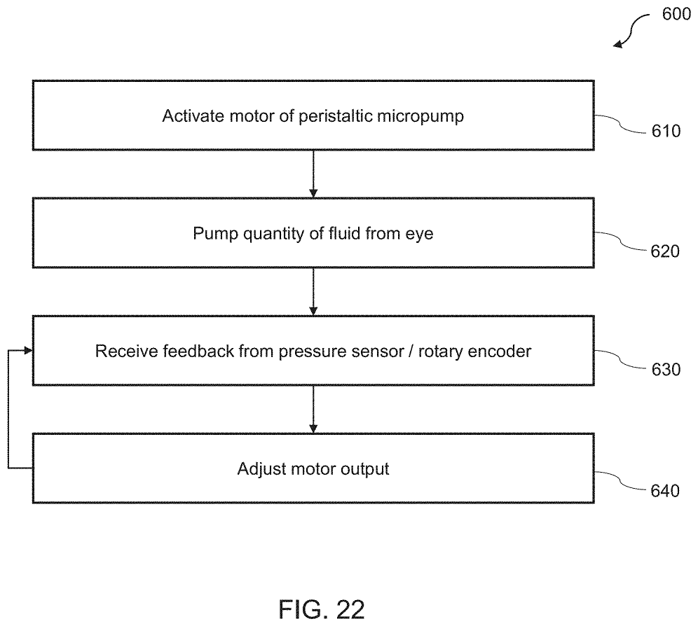

FIG. 22 depicts a method 600 of pumping a fluid (e.g., aqueous humor) from a patient's eye in order to reduce and/or regulate the patient's intraocular pressure (IOP). One or more steps of the method described can be carried out by a micropump assembly 110 as described above. In step 610, a motor of a micropump is activated to actuate a pump mechanism comprising a compressing member and a compressible fluid chamber. The motor rotates the compressing member about an axis in a circular motion, with the compressing member compressing a membrane of the fluid chamber against a hard outer ring. The fluid chamber is in communication with the patient's eye such that the micropump displaces fluid from inside the eye to the exterior of the eye. In step 620, the motor continues to rotate to pump a quantity of fluid from inside the eye, thereby reducing the IOP. The micropump may be controlled by an ASIC configured to control the output of the motor. The ASIC may control the output of the motor to displace a predetermined amount of fluid from the eye, to pump fluid at a predetermined flow rate, to operate the motor at a rotational speed, or some combination of these parameters.

In step 630, the ASIC receives feedback from a pressure sensor and/or a rotary encoder, and in step 640, the ASIC adjusts output of the motor based on the received feedback. For example, the feedback from the pressure sensor may include an electrical signal indicating a pressure measurement. The pressure sensor can be in fluid communication with an inlet of the fluid chamber to measure the fluid pressure from a source of the micropump, such as the patient's eye. The ASIC receives the pressure measurement and adjusts motor output according to a protocol. For example, the ASIC may be configured to execute computer instructions to maintain IOP at a particular pressure. When the pressure sensor measures a pressure that exceeds a threshold, the ASIC controls the motor to pump a particular quantity of fluid from the patient's eye. If the pressure measurement falls below a threshold, the ASIC does not activate the motor, or decreases the output of the motor.

In another example, the ASIC executes instructions to deliver an amount of a pharmaceutical agent to the patient. The ASIC activates the motor to rotate and receives feedback signals from the rotary encoder indicating the rotational position of the motor and compressing member. The ASIC controls the motor to rotate until the rotary encoder indicates that the motor is at a predetermined rotational position corresponding to an amount of pharmaceutical agent delivered to the patient.

In another example, the fluid chamber includes a circular portion and a non-circular portion, as described above. When a positive pressure differential is present across the fluid chamber (e.g., when IOP is relatively high), fluid may flow freely through the fluid chamber even without pumping. The motor and compressing member can be used to control the flow rate of fluid by controlling the motor to position the compressing member at a location on the non-circular portion that corresponds to a particular flow rate. To allow fluid to freely flow through the fluid chamber, the ASIC controls the motor to position the compressing member at a location on the non-circular portion at which the fluid chamber is least compressed, or uncompressed. To halt flow of fluid through the fluid chamber, the ASIC controls the motor to position the compressing member at a position along the circular portion of the fluid chamber such that the fluid chamber is fully compressed by the compressing member, thereby restricting flow of fluid through the fluid chamber.

In another example, the ASIC can include instructions to periodically pump fluid through the fluid chamber in order to prevent or remove clogs within the fluid chamber. For example, even when the IOP is below a threshold amount, or when a positive pressure gradient exists across the fluid chamber such that fluid is freely flowing without pumping, the ASIC may periodically activate the motor to compress the fluid chamber along its circumference to dislodge build-up of material and remove clogs.

It will be understood that various modifications can be made to the embodiments described above without departing from the material of the present disclosure. For example, although an ASIC is described as controlling the operation of the micropump assembly, other components and/or circuitry can be used to control operation of the micropump. For example, the micropump could include analog circuitry configured to control aspects of the micropump. The analog circuitry could function alone, or in combination with one or more microprocessors, field-programmable gate arrays (FPGA's), or any other appropriate analog or digital circuitry. Additionally, aspects of the different embodiments described above can be combined, even if the combinations are not explicitly shown in the drawings. For example, a micropump assembly can include a drug reservoir 119 as in FIG. 10 and a pressure sensor as in FIG. 9, in some embodiments. In another embodiment, a micropump assembly can include a spring-loaded rotor 136 as in FIG. 9 along with the drug reservoir 119 shown in FIG. 10. Additionally, any of the micropump assemblies described above can include a non-circular fluid chamber, as shown in FIG. 8.

The peristaltic pump may incorporate other components, including but not limited to gears, belts, additional rollers, an electrostatic motor, a pinch valve, a flow controller, a pressure sensor, a pressure regulator, one or more rotor bearings, an encoder, a microcontroller, and a motor coupling to drive the roller or rollers. The peristaltic pump may be a microelectromechanical systems (MEMS) device or incorporate MEMS components, or it may be a macroscopic device assembled from macroscopic components.

The ASIC can include one or more processing components and one or more memory components. The ASIC can be configured to execute computer code according to one or more programming protocols. In some example embodiments, one or more of the ASIC functions described above are executed by a computer program written in, for example, C, C Sharp, C++, Arena, HyperText Markup Language (HTML), Cascading Style Sheets (CSS), JavaScript, Extensible Markup Language (XML), asynchronous JavaScript and XML (Ajax), and/or any combination thereof.

Persons skilled in the art will recognize that the devices, systems, and methods described above can be modified in various ways not explicitly described or suggested above. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A peristaltic pump assembly, comprising:
a fluid chamber comprising a fluid channel configured to allow a fluid to pass therethrough, wherein the fluid chamber comprises:
a hard outer portion comprising a bell-shaped groove on an inner surface of the hard outer portion; and
a flexible membrane attached to the hard outer portion and extending over the inner surface of the hard outer portion, wherein the bell-shaped groove and the flexible membrane define the fluid channel; and
a roller coupled to the fluid chamber and configured to deform the flexible membrane against the bell-shaped groove on the inner surface of the hard outer portion to collapse the fluid channel.

2. The peristaltic pump assembly of claim 1, wherein the flexible membrane and the bell-shaped groove of the hard outer portion are configured such that a maximum stress experienced by the flexible membrane while being deformed against the bell-shaped groove is below a fatigue limit of the flexible membrane.

3. The peristaltic pump assembly of claim 1, wherein the flexible membrane comprises a thickness between 25 um and 150 um.

4. The peristaltic pump assembly of claim 3, wherein the thickness of the flexible membrane is 50 um.

5. The peristaltic pump assembly of claim 1, wherein the roller comprises a fillet radius that is less than a radius of the bell-shaped groove.

6. The peristaltic pump assembly of claim 5, wherein a thickness of the flexible membrane is less than the fillet radius of the roller.

7. The peristaltic pump assembly of claim 1, wherein the flexible membrane is attached to the hard outer portion by an adhesive.

8. The peristaltic pump assembly of claim 1, wherein the flexible membrane is attached to the hard outer portion by a laser weld.

9. The peristaltic pump assembly of claim 1, wherein the flexible membrane is formed to include a camber.

10. The peristaltic pump assembly of claim 1, wherein the flexible membrane comprises silicone rubber.

11. The peristaltic pump assembly of claim 1, wherein the hard outer portion comprises an annular shape.

12. The peristaltic pump assembly of claim 1, wherein the bell-shaped groove comprises at least one of a Gaussian curve, a symmetric spline, a sinusoidal curve, or a mirrored biarc.

13. The peristaltic pump assembly of claim 1, wherein the bell-shaped groove comprises an inflection point between a concave portion of the bell-shaped groove and a convex portion of the bell-shaped groove.

14. The peristaltic pump assembly of claim 1, wherein the flexible membrane further includes a coating positioned over an outer surface of the flexible membrane, wherein a coefficient of friction of the coating is less than a coefficient of friction of the outer surface of the flexible membrane.

15. A method of assembling a peristaltic pump assembly, comprising:
assembling a fluid chamber, wherein assembling the fluid chamber comprises:
providing a hard outer portion comprising a bell-shaped groove on an inner surface of the hard outer portion; and
attaching a flexible membrane to the hard outer portion such that the flexible membrane extends over the inner surface of the hard outer portion, and such that the flexible membrane and the bell-shaped groove of the hard outer portion define a fluid channel; and
coupling a roller assembly comprising a roller to the fluid chamber such that the roller is configured to pass over the flexible membrane to deform the flexible membrane against the bell-shaped groove of the hard outer portion.

16. The method of claim 15, wherein the flexible membrane and the bell-shaped groove of the hard outer portion are configured such that a maximum stress experienced by the flexible membrane while being deformed against the bell-shaped groove is below a fatigue limit of the flexible membrane.

17. The method of claim 15, further comprising forming a roller fillet comprising a fillet radius that is less than a radius of the bell-shaped groove.

18. The method of claim 15, wherein attaching the flexible membrane to the hard outer portion comprises attaching the flexible membrane to the hard outer portion using an adhesive.

19. The method of claim 15, wherein attaching the flexible membrane to the hard outer portion comprises attaching the flexible membrane to the hard outer portion using a laser weld.

20. The method of claim 15, further comprising forming the flexible membrane to include a camber.

21. The method of claim 15, wherein the bell-shaped groove comprises at least one of a Gaussian curve, a symmetric spline, a sinusoidal curve, or a mirrored biarc.

22. The method of claim 15, wherein the bell-shaped groove comprises an inflection point between a concave portion of the bell-shaped groove and a convex portion of the bell-shaped groove.

23. The method of claim 15, wherein the flexible membrane further includes a coating positioned over an outer surface of the flexible membrane, wherein a coefficient of friction of the coating is less than a coefficient of friction of the outer surface of the flexible membrane.

24. A peristaltic pump assembly, comprising:
an annular fluid chamber comprising:
a hard ring comprising a concave groove on an inner surface of the hard ring; and
a membrane attached to the hard ring and extending over the inner surface of the hard ring to form a fluid channel comprising a curved cross-section; and
a roller assembly coupled to the annular fluid chamber comprising a roller configured to deform the membrane against the concave groove on the inner surface of the hard ring to collapse the fluid channel.

25. The peristaltic pump assembly of claim 24, wherein the membrane and the concave groove of the hard ring are configured such that a maximum stress experienced by the membrane while being deformed against the concave groove is below a fatigue limit of the membrane.

26. The peristaltic pump assembly of claim 24, wherein at least a portion of the concave groove comprises a circular arc.
